(19) United States
(12) Patent Application Publication
(10) Pub. No.: US 2007/0270969 A1
(43) Pub. Date: Nov. 22, 2007

(54) WELDED-WOVEN MATERIALS

(76) Inventor: Steven R. Schmid, Lakeville, IN (US)

Correspondence Address: HANLEY, FLIGHT & ZIMMERMAN, LLC, 150 S. WACKER DRIVE, SUITE 2100, CHICAGO, IL, 60606, US

(21) Appl. No.: 11/748,875

(22) Filed: May 15, 2007

Related U.S. Application Data
(60) Provisional application No. 60/800,955, filed on May 17, 2006.

(51) Int. Cl. A61F 2/44

(52) U.S. Cl. 623/17.11; 623/901; 156/81

(57) ABSTRACT

Three-dimensional welded-woven materials are disclosed. In particular, an orthopedic implant comprises a welded-woven material to provide lubrication and wear resistance.

### RELATED APPLICATION

This patent application claims priority pursuant to 35 U.S.C. §119 of U.S. Provisional Patent Application Ser. No. 60/800,955 filed on May 17, 2006, hereby incorporated by reference in its entirety.

### FIELD OF THE DISCLOSURE

This disclosure relates generally to welded-woven materials and, more particularly, to welded-woven orthopedic implants.

### BACKGROUND

With the great advances in medical care in the past few decades, life expectancies and the quality of life have been considerably increased, but so have expectations that effective health care treatments exist for all maladies. One reason for the improvements in life expectancy and quality of life is the great success of orthopedic implants developed during the past several decades. Hip, knee, shoulder, elbow, ankle and spine implants have resulted in millions of people worldwide having increased physical activity and reduced pain. Such advances in medical technology have contributed to the continuing increase in life expectancy, and as the post-World War II generation continues to age, the number of orthopedic operations will increase.

The current technology has provided orthopedic implants that are invasive and painful due to the total replacement of the joint. Even for seniors, the availability of orthopedic treatments requires an extreme level of pain before a surgeon will resort to implantation. This is understandable because although modern implants work very well, they require painful surgery and rehabilitation. Additionally, the possibility of premature implant failure is always a concern. The subsequent replacement of a total joint replacement is more invasive and painful than the initial replacement.

### BRIEF DESCRIPTION OF THE DRAWINGS

FIGS. 1a and 1b are schematic illustrations of known woven architectures commonly used in three-dimensional composites.

FIG. 2 is an illustration of a knee joint that includes an example orthopedic implant.

FIG. 3 is a flowchart of an example method to make a three-dimensional welded-woven material.

### DETAILED DESCRIPTION

Conventional three-dimensional (3D) woven materials have been used for aerospace applications for over twenty years. However, their unique characteristics and advantages have not been used for medical products, in general, and orthopedic implants, in particular. This type of materials is described in detail by Mouritz, A. P., Bannister, M. K., Falzon, P. J., and Leong, K. H., “Review of Applications for Advanced Three-Dimensional Fibre Textile Composites,” Composites: Part A, v. 30, 1999, pp. 1445-1461, and Kamiya, R., Cheeseman, B. A., Popper, P., and Chou, T.-W., “Some Recent Advances in the Fabrication and Design of Three-Dimensional Textile Preforms: A Review,” Composites Science and Technology, v. 60, 2000, pp. 33-47, the entire contents and disclosures hereby incorporated by reference. Some of the attractive features of this type of materials includes:

3D weaving can produce near-net-shape products or performs.

Any metal, polymer, or ceramic that can be produced in fiber form can be woven, including FDA-approved materials and materials of current interest in the orthopedic industry.

The through-thickness properties of 3D woven materials can be tailored for particular applications.

3D woven composites have a higher delamination resistance and impact damage tolerance than two-dimensional laminated composites.

3D weaving can be controlled automatically so that the production and preform quality are very high.

Different materials can be blended into a fiber prior to weaving. Indeed, most clothing involves blends of polymers or of polymers and natural fibers such as cotton or linen.

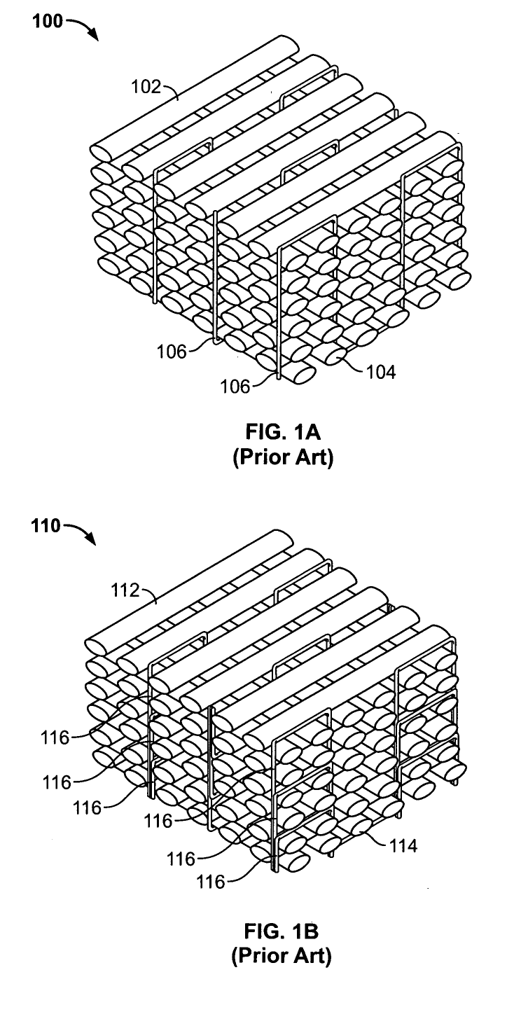

Different fibers can be woven in a 3D weave. FIGS. 1a and 1b are schematic illustrations of known woven architectures commonly used in 3D composites. FIG. 1a illustrates an orthogonal weave 100 having weft tows 102, warp tows 104 and binder yarns 106. FIG. 1b illustrates a layer-interlock weave 110 having weft tows 112, warp tows 114 and binder yarns 116. As can be readily seen from FIGS. 1aand 1b, some or all of the warp tows 104, 114, the weft tows 102, 112, and the binder yarns 106, 116 may be produced from different materials and blended to form a multi-material woven composite.

The size of the weave can be varied to allow for changes in structure in such a material.

The amount of different materials can be varied throughout a bulk woven volume, yielding true functionally graded materials.

These characteristics of 3D woven materials provide a large number of new capabilities to implant researchers. However, Mouritz, A. P., Bannister, M. K., Falzon, P. J., and Leong, K. H., “Review of Applications for Advanced Three-Dimensional Fibre Textile Composites,” Composites: Part A, v. 30, 1999, pp. 1445-1461, suggest that since 3-D weaves have lower mechanical properties than laminated composites, their proliferation has been impeded.

The 3-D woven materials illustrated in FIG. 1 are known to those skilled in the art. However, it is often desired to increase the stiffness, strength, fatigue properties, lubrication effectiveness or resilience of the woven material. This can be accomplished by selectively welding some or all of the fibers or tows.

Selective welding can take place by using a combination of polymer fibers wherein one polymer fiber has a lower melting temperature than the others in the woven material. When the example 3-D woven material is either heated, subjected to ultrasonic welding, or heated by a light energy source such as, for example, a laser, the low-melting temperature polymer will weld to adjacent polymer fibers.

Alternatively, laser welding may be used to locally weld fibers. Such laser welding can be accomplished through the thickness of the example 3-D woven material via selected points on the surface of the material, or it can be accomplished at selected fibers.

Laser energy can be provided at a wavelength so that the polymer fibers are transparent. Also, transparent fibers may be made of a glass that transmits light energy. However, polymer fibers may not be transparent if they contain a toner or other object that absorbs light energy. The toner can be carbon soot, ink or other materials that absorb light. The polymer fibers may contain a metal powder, such as, for example, a micro-scale or a nano-scale metal powder. Alternatively, the fibers may be made of a light radiation absorbing material such as, for example, graphite, metal or ceramic.

A biocompatible toner such as Clearweld™ from Gentex Corporation (www.gentex.com) can be used to preserve the biocompatibility of the welded-woven materials. This is especially useful for in-vivo applications.

The example woven material may have a portion of its binder yarns, weft tows, and/or warp tows (e.g., such as the binder yarns 106, 116, weft tows 102, 112, and/or warp tows 104, 114 illustrated in FIGS. 1a and 1b) either doped with or coated by the toner. The doping can be done for most polymers before spinning the fibers. Alternatively, the selected polymer fibers can be coated before weaving. An example 3D structure or woven material is then woven as desired. The porosity of the example 3D woven material can be made to vary throughout the material.

If desired, the polymer fibers can be loaded in a suitable fixture to place the weave in tension or compression, either over the entire volume, or localized. The example 3-D woven material can be loaded to achieve complex stress or strain distributions.

Once the example woven material has been stressed or strained as desired, it is exposed to a laser or other light energy source. The light energy causes polymer fibers that contain or are coated by toner to heat preferentially, causing them to weld to adjacent polymer fibers. If a fiber has no toner, it doesn't heat appreciably and will not weld. Thus, selective welding occurs.

Typically, the light source or laser is set at a wavelength of 940-1000 nm, and the light energy can be scanned across a volume. Such laser welding can be used to bond selected polymer fibers to increase the strength, but more importantly, to increase the recovery and resilience of the woven material.

The selective laser welding approach may be used with any woven thermoplastic including, but not limited to, polyester, polyethylene, acrylics such as, for example, polymethyl methacrylate, polytetrafluoroethylene (Telon®), ketones such as, for example, polyether etherketone and polyaryl etherketone, and hydrogrels such as, for example, polyvinyl alcohol (PVA).

Preloading the example 3D woven material during welding is important because, subsequent to the laser welding, the material will then tend to recover toward the structural condition of the weave at welding when unloaded. For example, an example 3D woven material can be stretched to a shape and the welding of selected interfaces in the material can lock or fix the material in that condition. The material will deform under load, but when the load is released, the example 3D woven material will return to its as-welded shape. Thus, the resilience of the material can be substantially increased by this method.

In another approach, the example 3D welded-woven material can be used in injection molding to produce a composite material. Subsequent laser heating of the material will melt and fracture stressed welds, causing the internal stresses to redistribute to the matrix. Therefore, a compressive residual stress can be imparted to the polymer that acts to improve the polymer's strength.

This class of material has been termed “hybrid welded-woven” materials which are produced by the novel method of making the example 3D welded-woven materials.

The density of welds can be modified within an example 3-D woven structure by changing the concentration of doped or coated fibers within an area of the volume, or else by modifying the laser or light energy exposure according to location. Thus, the selective welding accentuates the ability of the woven material to achieve functionally-graded mechanical properties.

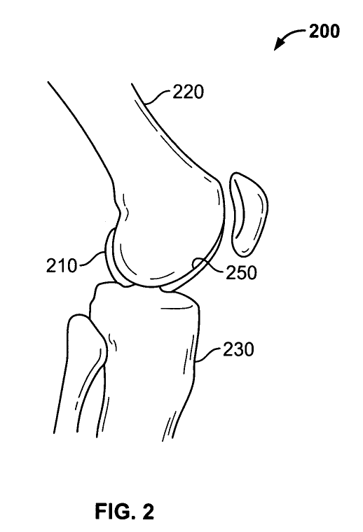

FIG. 2 is an illustration of a knee joint 200 that includes an example implant 210 having the material properties and capabilities of three-dimensional weaving. A healthy knee joint includes elastic tissue or cartilage. In FIG. 2 the knee joint 200 includes the example implant 210, which is disposed between a femur 220 and a tibia 230. The example implant 210 has been substituted for previously damaged or worn cartilage and may extend into and be connected to the porous trabecular bone 250 of the femur 220 or the tibia 230.

Tribology of Woven Materials. Described herein are topics pertinent to the opportunities emanating from the use of welded-woven materials. Although some background is provided on tribology issues in orthopedics, more complete reference texts are Hamrock, B. J., Schmid, S. R., and Jacobson, B. O., Fundamentals of Fluid Film Lubrication, 2^{nd} ed. New York, Marcel-Dekker, 2004, 2005; Bhushan, B., Introduction to Tribology, New York, Wiley & Sons, 2002; or Szeri, A. Z., Fluid Film Lubrication. Cambridge, Cambridge University Press, 1998, the entire contents and disclosures hereby incorporated by reference.

One of the most fundamental concepts of lubricated conjunctions is that of “Regimes of Lubrication”, described in practically any modern tribology textbook. The regimes of lubrication describe the media through which load is transferred between surfaces—through a pressurized lubricant film, through opposing surface asperities or a combination of these effects—and are commonly defined through a film parameter, Λ, defined by

$$Λ = \frac{h}{{R}_{qa}^{2} + {R}_{qb}^{2}} = \frac{h}{σ}$$

where h is the film thickness, R_{qa} and R_{qb} are the surface roughnesses of the two surfaces in contact, and σ is the composite roughness. If Λ is less than one, it is generally considered to be a circumstance of boundary lubrication, where all of the load transfer occurs across asperities with the possible presence of a molecularly thick layer of a boundary species. If Λ is greater than 3, then full film lubrication occurs, where the load between asperities is transferred across a pressurized lubricant, and asperities are rarely in contact. Sometimes full film lubrication is separated into two regimes, that of thin film (where 3≦Λ≦10) and thick film (Λ≧10), although this distinction is not particularly useful for orthopedic implants.

The effect of film parameter on friction and fatigue wear is dramatic, and is especially strong in the boundary and mixed regimes. Small increases in the film thickness (and hence film parameter) will lead to very large decreases in wear rates. Thus, there is an advantage in providing an implant such as, for example, the example implant 200 in FIG. 2, that will maximize the entrained fluid film and film parameter

The film parameter for natural joints is higher than for artificial joints. See Hamrock, B. J., Schmid, S. R., and Jacobson, B. O., Fundamentals of Fluid Film Lubrication, 2^{nd} ed. New York, Marcel-Dekker, 2004, the entire contents and disclosure hereby incorporated by reference, which investigates the reasons for this disparity in detail. However the following reasons are contributory:

An important mechanism that occurs in natural cartilage is the generation of hydrodynamic films through exudation of lubricant from the cartilage itself. See McCutcheon, C. W., “The Frictional Properties of Animal Joints,” Wear, v. 5, 1962, p. 1; Mow, V. C., Mak, A. F., and Lai, W. M., “Viscoelastic Properties pf Proteoglycan Subunits and Aggregates in Varying Solution Concentrations,” J. Biomech., v. 17, 1984, pp. 181-222, and Fisher, J., “Biomedical Applications” in Bhushan, B., Modern Tribology Handbook. Boca Raton: CRC Press, 2001, the entire contents and disclosures hereby incorporated by reference. This is referred to as “weeping film” lubrication or “percolation lubrication”. See Lo, S.-W., “A study of flow phenomena in mixed lubrication regime by porous media model,” J. Tribology, v. 116, 1994, pp. 640-647, the entire contents and disclosure hereby incorporated by reference. Very little fluid is needed for effective lubrication. For example, in a total knee replacement (TKR) a drop of fluid 0.1 mm in diameter is sufficient to completely separate the surfaces. Cartilage apparently provides just enough fluid to attain such lubrication conditions, while current artificial implants do not provide such lubrication.

As discussed by Dowson, D., and Jin, Z. M., “Micro-elastohydrodynamic lubrication of Low Elastic Modulus Solids on Rigid Substrates,” J. Phys. D: Appl. Phys., Frontiers of Tribology, v. 25, 1992, pp. 133-140, the entire contents and disclosure hereby incorporated by reference, the asperities of the soft cartilage can be flattened dynamically, effectively yielding a higher film parameter with the same film thickness by dynamically reducing surface roughness.

Dowson, D., “Lubrication of Joints: A: Natural Joints”, in An Introduction to the Bio-Mechanics of Joints and Joint Replacements, D. Dowson and V. Wright (eds.), Mechanical Engineering Publications, 1981, pp. 120-133, the entire contents and disclosure hereby incorporated by reference, analyzed the human hip and knee in some detail and concluded that any hydrodynamic films developed during the swing phase of walking can be preserved by a combination of entraining and squeeze-film action during the stance phase.

A natural knee joint represents a situation of soft elastohydrodynamic lubrication (EHL), or at worst, soft EHL of a coating on a hard substrate. See Dowson, D., and Yao, J. Q., “Elastohydrodynamic Lubrication of Layered Solids in Elliptical Contacts. Part 2: Film Thickness Analysis,” Proc. Inst. Mech. Eng. Part J: J. Eng. Trib., v. 208, 1994, pp. 43-52. Artificial implants are examples of hard EHL. Hard EHL can be very successful in tribological situations, but only when the lubricating fluid has good high-pressure rheology. This is evidently not the case for synovial fluid (see Schey, J. A., “Systems View of Optimizing Metal-on-Metal Bearings,” Clin. Orthop. Rel. Res., 1996, pp. S115-S127, the entire contents and disclosure hereby incorporated by reference), so that soft EHL results in thicker lubricant films than hard EHL in vivo.

The percolation lubrication mechanisms present in natural joints can be duplicated using micro-porous hydrophilic materials, such as 3-D welded-woven polymers. These materials have the desirable trait of releasing water when subjected to pressure, and because of increased resilience, reabsorb the water when unloaded.

Welded-woven materials are beneficial for cartilage replacement and other orthopedic and soft tissue applications. Woven materials have been investigated for cartilage applications—see for example Moutos, F. T., Freed, L. E., and Guilak, F., “A biomimetic three-dimensional woven composite scaffold for functional tissue engineering of cartilage”, Nature Materials, v. 6, February 2007, the entire contents and disclosure hereby incorporated by reference. While mechanical properties of such woven materials can be made to approximate cartilage, the long-term survivability has not been demonstrated. By incorporating a welded component to the microstructure, the lubricating effectiveness of the material will be improved and will result in a longer-lived cartilage replacement.

Welded-woven UHMWPE structures are good candidates for wear surfaces.

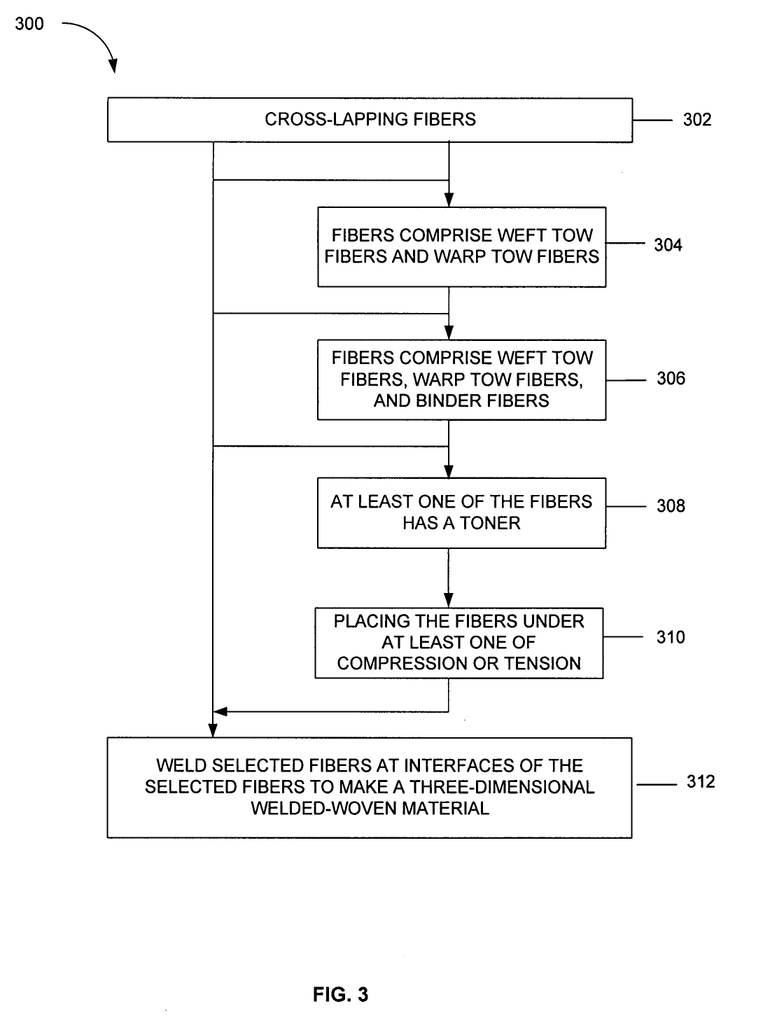

FIG. 3 is a flowchart of an example method 300 to make a 3-D welded-woven material. At block 302, the example method 300 includes providing cross-lapping fibers (e.g., the weft tows 102, the warp tows 104, or the binder yarns 106 of the orthogonal weave 100 in FIG. 1a or the weft tows 112, the warp tows 114, or the binder yarns 116 of the layer-interlock weave 110 in FIG. 1b). The example method 300 includes options illustrated at blocks 304, 306, 308 and 310. At block 304, the fibers maybe weft tow fibers (e.g., the weft tows 102 in FIG. 1a or the weft tows 112 in FIG. 1b) and warp tow fibers (e.g., the warp tows 104 in FIG. 1a or the warp tows 114 in FIG. 1b). Optionally, the fibers are weft tow fibers, warp tow fibers, and binder fibers (e.g., the weft tows 102, the warp tows 104, and the binder yarns 106 in FIG. 1a or the weft tows 112, the warp tows 114, and the binder yarns 116 in FIG. 1b), (block 306).

At block 308, at least one of the fibers (e.g., the weft tows 102, the warp tows 104, or the binder yarns 106 in FIG. 1a or the weft tows 112, the warp tows 114, or the binder yarns 116 in FIG. 1b) may have a toner (e.g., a toner such as, for example, the biocompatible toner Clearweld™). Optionally, the fibers (e.g., the weft tows 102, the warp tows 104, or the binder yarns 106 of the orthogonal weave 100 in FIG. 1a or the weft tows 112, the warp tows 114, or the binder yarns 116 of the layer-interlock weave 110 in FIG. 1b) may be under at least one of compression or tension, (block 310).

The example method 300 then includes welding selected fibers (e.g., the weft tows 102, the warp tows 104, or the binder yarns 106 in FIG. 1a or the weft tows 112, the warp tows 114, or the binder yarns 116 in FIG. 1b) at interfaces of the selected fibers to make a three-dimensional welded-woven material, (block 312). The three-dimensional welded-woven material may be used in numerous applications including orthopedic implant applications such as, for example, the example implant 200 in FIG. 2.

Example methods of making welded-woven materials and welded-woven materials are described with reference to the flowchart illustrated in FIG. 3. However, persons of ordinary skill will readily appreciate that other methods of implementing the example method may alternatively be used. For example, the order of execution of the blocks may be changed, and/or some of the blocks described may be changed, eliminated, or combined

Although certain example methods and articles have been described herein, the scope of coverage of this patent is not limited thereto. On the contrary, this patent covers all methods, apparatus and articles of manufacture fairly falling within the scope of the appended claims either literally or under the doctrine of equivalents.

What is claimed is:

1. A three-dimensional welded-woven material, comprising cross-lapped fibers and welds at selected interfaces of the cross-lapped fibers.

2. A material as claimed in claim 1, wherein the fibers were under at least one of compression or tension prior to welding.

3. A material as claimed in claim 1, wherein the welded-woven material comprises a woven polymer.

4. A material as claimed in claim 3, wherein the fibers are under at least one of compression or tension prior to welding.

5. A material as claimed in claim 3, wherein the woven polymer comprises at least one of polyethylene, polyether etherketone, polyaryl etherketone, polymethylm methacrylate, polytetrafluoroethylene, polyester, or a hydrogel.

6. A material as claimed in claim 1, wherein the welded-woven material includes a tribological material.

7. A material as claimed in claim 6, wherein the tribological material comprises at least one of polyethylene, polytetrafluoroethylene, polyaryl etherketone, carbon, or a hydrogel.

8. A material as claimed in claim 6, wherein the tribological material retains a fluid and releases the fluid when subjected to pressure.

9. A material as claimed in claim 8, wherein the fluid is synovial fluid.

10. A material as claimed in claim 6, wherein the tribological material includes tribopolymers.

11. A material as claimed in claim 1, wherein the welds are laser welds.

12. A material as claimed in claim 1, wherein the welds are electron beam welds.

13. A material as claimed in claim 1, wherein the welds are fusion bonds.

14. A material as claimed in claim 1, wherein selected fibers of the cross-lapped fibers include a toner.

15. A material as claimed in claim 14, wherein the toner is a biocompatible toner.

16. A material as claimed in claim 14, wherein the toner was at least one of doped or coated to the selected fibers.

17. A material as claimed in claim 1, wherein selected fibers of the cross-lapped fibers comprise a light radiation absorbing material.

18. A material as claimed in claim 17, wherein the light radiation absorbing material is at least one of graphite, metal or ceramic.

19. A material as claimed in claim 1, wherein the material is an orthopedic implant.

20. A material as claimed in claim 19, wherein the orthopedic implant comprises at least one of an orthopedic hip implant, orthopedic knee implant, orthopedic shoulder implant, orthopedic elbow implant, orthopedic ankle implant, orthopedic finger implant, or orthopedic spine disc implant.

21. A material as claimed in claim 19, wherein the implant is to replace elastic tissue.

22. A material as claimed in claim 21, wherein the elastic tissue is cartilage.

23. A material as claimed in claim 1, wherein the porosity of the material is not uniform throughout the material.

24. A material as claimed in claim 1, wherein the welds are located non-uniformly throughout the material.

25. A material as claimed in claim 1, wherein the cross-lapped fibers include fibers made of at least one of carbon, graphite, or metal.

26. A material as claimed in claim 25, wherein the cross-lapped fibers further include fibers of a light energy transmitting material.

27. A material as claimed in claim 26, wherein the light energy transmitting material is at least one of a polymer or a glass.

28. A material as claimed in claim 1, wherein the cross-lapped fibers include fibers made from a polymer containing a metal powder.

29. A material as claimed in claim 28, wherein the metal powder is at least one of a micro-scale metal powder or a nano-scale metal powder.

30. An implant for elastic tissue, comprising a welded-woven material to provide lubrication and wear resistance.

31. An implant as claimed in claim 30, wherein the welded-woven material is to replace elastic tissue.

32. An implant as claimed in claim 30, wherein the elastic tissue is cartilage.

33. An implant as claimed in claim 30, wherein the implant may be connected to trabecular bone.

34. An implant as claimed in claim 30, wherein the welded-woven material includes a tribological material.

35. An implant as claimed in claim 30, wherein the welded-woven material was under at least one of compression or tension prior to welding.

36. An implant as claimed in claim 30, wherein the welded-woven material comprises a woven polymer.

37. An implant as claimed in claim 36, wherein the woven polymer comprises at least one of wherein the woven polymer comprises at least one of polyethylene, polyether etherketone, polyaryl etherketone, polymethylm methacrylate, polytetrafluoroethylene, polyester, or a hydrogel.

38. An implant as claimed in claim 30, wherein the welded-woven material comprises a tribological material that retains a fluid and releases the fluid when subjected to pressure.

39. An implant as claimed in claim 38, wherein the fluid is synovial fluid.

40. An implant as claimed in claim 39, wherein the tribological material includes tribopolymers.

41. An implant as claimed in claim 30, wherein the welded-woven material includes laser welds.

42. An implant as claimed in claim 30, wherein the welded-woven material includes selected fibers having a toner.

43. An implant as claimed in claim 42, wherein the toner is a biocompatible toner.

44. An implant as claimed in claim 43, wherein the biocompatible toner was at least one of doped or coated to the selected fibers.

45. An implant as claimed in claim 30, wherein the implant is an orthopedic implant comprising at least one of an orthopedic hip implant, orthopedic knee implant, orthopedic shoulder implant, orthopedic elbow implant, orthopedic ankle implant, orthopedic finger implant, or orthopedic spine disc implant.

46. A method to make a three-dimensional welded-woven material, the method comprising:
cross-lapping fibers including selected fibers treated by at least one of doping or coating, and
welding the selected fibers at interfaces of the selected fibers to make the three-dimensional welded-woven material.

47. A method as claimed in claim 46, wherein the welding is laser welding.

48. A method as claimed in claim 47, wherein the laser welding is within a wavelength range of approximately 940-1000 nm.

49. A method as claimed in claim 46, wherein a toner is used in the doping or coating.

50. A method as claimed in claim 49, wherein the toner is a biocompatible toner.

51. A method as claimed in claimed 46, wherein the welded-woven material is to provide lubrication and wear resistance.

52. A method as claimed in claim 46, wherein fibers of the welded-woven material are to be connected to trabecular bone.

53. A method as claimed in claim 46, wherein the welded-woven material comprises a woven polymer.

54. A method as claimed in claim 53, wherein the woven polymer comprises at least one of polyethylene, polyether etherketone, polyaryl etherketone, polymethylm methacrylate, polytetrafluoroethylene, polyester, or a hydrogel.

55. A method as claimed in claim 46, wherein the welded-woven material is a tribological material to retain and release a fluid when subjected to pressure.

56. A method as claimed in claim 55, wherein the fluid is synovial fluid.

57. A method as claimed in claim 55, wherein the tribological material includes tribopolymers.

58. A method to make a three-dimensional welded-woven material, the method comprising:
cross-lapping a combination of at least one of light transmitting polymer fibers and light absorbing polymer fibers or electrically conductive polymer fibers and nonconductive polymer fibers, and
welding selected fibers at interfaces of the selected fibers to make the three-dimensional welded-woven material.

59. A method as claimed in claim 58, further comprising placing the fibers under at least one of compression or tension prior to welding.

60. A method as claimed in claim 58, wherein the welding is electron-beam welding.

61. A method as claimed in claim 58, wherein the welding is fusion bonding.

62. A method as claimed in claim 58, wherein the welded-woven material comprises a woven polymer.

63. A method as claimed in claim 62, wherein the woven polymer comprises at least one of polyethylene, polyether etherketone, polyaryl etherketone, polymethylm methacrylate, polytetrafluoroethylene, polyester, or a hydrogel.

64. A method as claimed in claim 58, wherein the welded-woven material is a tribological material to retain and release a fluid when subjected to pressure.

65. A method as claimed in claim 64, wherein the fluid is synovial fluid.

66. A method as claimed in claim 64, wherein the tribological material includes tribopolymers.
